(19) United States
(12) Patent Application Publication
(10) Pub. No.: US 2009/0062898 A1
(43) Pub. Date: Mar. 5, 2009

(54) Catheter system for bifurcated coronary vessel

(76) Inventor: Gladwin S. Das, St. Paul, MN (US)

Correspondence Address: CYR & ASSOCIATES, P.A., 605 U.S. Highway 169, Suite 300, Plymouth, MN, 55441, US

(21) Appl. No.: 12/215,371

(22) Filed: Jun. 25, 2008

Related U.S. Application Data
(60) Provisional application No. 60/937,097, filed on Jun. 25, 2007.

(51) Int. Cl. A61F 2/84; A61M 25/10

(52) U.S. Cl. 623/1.11; 604/101.01

(57) ABSTRACT

Devices and methods for treating bifurcated body vessels, including stenosed, bifurcated coronary arteries. Methods can include advancing a guide catheter to the stenosed bifurcated region, advancing first and second guide wires through the guide catheter into separate vessel regions past the bifurcation, and then withdrawing the guide catheter. The method proceeds by advancing a balloon catheter having a bifurcated stent mounted to a Y-shaped distal balloon region over the guide wires, and positioning the bifurcated stent proximal of and distal to the bifurcation. Inflating the balloons dilates the stenosed region and expands the bifurcated stent. Kits provided by the present invention can include two guide wires, a guide catheter, a balloon catheter having a Y-shaped distal balloon region, and a bifurcated stent configured to be carried over the distal balloon region.

### CROSS-REFERENCE TO RELATED APPLICATIONS

The present application claims the benefit and priority of U.S. provisional patent application No. 60/937,097 filed on Jun. 25, 2007 and entitled CATHETER SYSTEM FOR BIFURCATED CORONARY VESSEL, the disclosure of which is hereby incorporated herein in its entirety by reference thereto.

### BACKGROUND OF THE INVENTION

1. Field of the Invention

The present inventions relate to medical devices and more particularly, the present inventions relate to apparatus and methods for treatment of bifurcated lumen with the body of a patient.

2. Background of the Related Art

Coronary arteries can become stenosed and narrowed over time, with deleterious results. The narrowing may be gradual, causing increasing angina, ultimate occlusion, and myocardial infraction. The narrowing maybe be gradual in part, followed a sudden rupture, clotting, and myocardial infarction. The myocardial infraction may lead to partial destruction of the heart muscle and sudden death.

Coronary artery stenosis has been addressed using several methods well known to those skilled in the art. One common method includes balloon angioplasty. In this method, a guide catheter may be introduced near the groin, advanced through the femoral artery, over the aortic arch, and further into or near the left or right coronary artery left ostium. A thin wire, a guide wire, can be advanced through the guide catheter and further through the selected right or left coronary artery, into the tortuous coronary vasculature of the heart, until the guide wire is placed past or across the stenosed region. The guide catheter can be removed at some point in this procedure.

A balloon angioplasty catheter can be threaded over the guide wire near the groin and advanced to the stenosed vessel region. The balloon on the distal region of the balloon catheter can be inflated, dilating or widening the stenosed vessel region. The balloon can then be deflated and withdrawn from the body.

In a large percent of cases, the dilated region soon narrows, due to restenosis. In order to reduce the occurrence of the restenosis, to maintain the patency of the dilated vessel region, a stent may be carried on the balloon, and expended into a larger diameter configuration against the vessel wall, as the balloon is inflated. The stent is generally a wire mesh, which retains its expanded configuration after the balloon is deflated and withdrawn from the vessel. Stents may be coated with a drug, e.g. paclitaxel, to further inhibit restenosis.

Simple guide catheters, guide wires, and stents may be used effectively on simple stenosed regions. Coronary blood vessel repeatedly branch or bifurcate from the larger coronary blood vessels down to the much smaller and more numerous blood vessels. In some cases, the branched or bifurcated regions are stenosed. In one example, a coronary artery region upstream of a branch or bifurcation is stenosed along with two vessel downstream regions. It may be difficult to deliver three balloon angioplasty catheters and stents across the three stenosed regions. At best, if successful, it may take much longer to successfully dilate the three regions using balloons and to place three stents, tying up catheter labs and driving up costs.

What would be advantageous are devices and systems for dilating stenosed bifurcated vessels. What would be desirable are devices and methods for more quickly delivering more than one guide wire, balloon, and stent to a bifurcated vessel region having more than one stenosed portion.

### SUMMARY OF THE INVENTION

Apparatus and methods in accordance with the present inventions may meet one or more of the above-referenced needs and will provide additional improvements and advantages that will be recognized by those skilled in the art upon review of the following disclosure and attached figures. The present inventions can provide apparatus and methods for treating a stenosed, bifurcated vessel region in a human body. The apparatus includes a first guide wire and a second guide wire. The bifurcation typically has a main branch, which includes the vessel approaching the bifurcation and a side branch. The first guide wire and the second guide wire may be placed in the main branch and the side branch respectively by a guide catheter. The guide catheter includes a first guide wire lumen sized to slidably receive the first guide wire, and the guide catheter includes a second guide wire lumen sized to slidably receive the second guide wire. The guide catheter is configured to be advanced through various bodily lumen to the bifurcation. The guide catheter may be further configured to facilitate placement of the first guide wire into the main branch and to facilitate placement of the second guide wire into the side branch before the guide catheter is withdrawn.

The apparatus may include a bifurcated stent for dilation and support of the vessels in the region of the bifurcation. The bifurcated stent may be positioned in the bifurcation by a balloon catheter. The balloon catheter may include at least a first balloon and a second balloon, and the bifurcated stent may be mounted to at least the first balloon and the second balloon. The balloon catheter, including the bifurcated stent mounted over at least the first balloon and the second balloon, is configured to advance over the first guide wire and the second guide wire. The bifurcated stent is positioned in the bifurcation by advancing the balloon catheter over the first guide wire and the second guide wire, whereupon the first balloon and the second balloon are inflated to dilate the blood vessel in the region of the bifurcation and to support the blood vessel with the bifurcated stent in the region of the bifurcation.

Methods in accordance with the present inventions can include advancing a guide catheter to the bifurcation, placing the first guide wire in the main branch of the bifurcation, placing the second guide wire in the side branch of the bifurcation, and then withdrawing the guide catheter. The method then proceeds by mounting a bifurcated stent to a plurality of balloons on a balloon catheter, advancing the balloon catheter over the first guide wire and the second guide wire, positioning the bifurcated stent at the bifurcation, dilating the bifurcated stent and dilating the vessels in the region of the bifurcation by inflating the plurality of balloons, deflating the plurality of balloons, and withdrawing the balloon catheter from the patient.

### BRIEF DESCRIPTION OF THE FIGURES

FIG. 1 illustrates a partial side view of an embodiment of a guide catheter in accordance with the present inventions;

FIG. 2 illustrates a partial side view of an embodiment of a kit including a first guide wire, a second guide wire, a guide catheter and a balloon catheter in accordance with the present inventions;

FIG. 3 illustrates a fragmentary, perspective view of an embodiment of a proximal portion of a guide catheter;

FIG. 4A illustrates a fragmentary, perspective view of an embodiment of a distal region of a guide catheter in accordance with the present inventions;

FIG. 4B illustrates an end view of an embodiment of a distal region of a guide catheter in accordance with the present inventions;

FIG. 5A illustrates a fragmentary, perspective view of another embodiment of a distal region of a guide catheter in accordance with the present inventions;

FIG. 5B illustrates an end view of another embodiment of a distal region of a guide catheter in accordance with the present inventions;

FIG. 6A illustrates a fragmentary, perspective view of another embodiment of a distal region of a guide catheter in accordance with the present inventions;

FIG. 6B illustrates an end view of another embodiment of a distal region of a guide catheter in accordance with the present inventions;

FIG. 7A illustrates a fragmentary, perspective view of another embodiment of a distal region of a guide catheter in accordance with the present inventions;

FIG. 7B illustrates an end view of another embodiment of a distal region of a guide catheter in accordance with the present inventions;

FIG. 8 illustrates a partial side view in cross-section of an embodiment of a distal region of a guide catheter in accordance with the present inventions;

FIG. 9 illustrates a partial perspective view in cross-section of another embodiment of a distal region of a guide catheter in accordance with the present inventions;

FIG. 10 illustrates a perspective view of a bifurcated Y-shaped stent having a main mesh tube coupled to a first mesh tube and a second mesh tube;

FIG. 11A illustrates a schematic side view of a an angioplasty balloon catheter delivery system which can be used to place the bifurcated stent of FIG. 10 over guide wires placed using the guide catheter of FIGS. 1A and 1B;

FIG. 11B illustrates a fragmentary, side, cross-sectional schematic view of the distal portion of the angioplasty balloon catheter having a flexible distal single shaft extending further distally into a main balloon and a side branch balloon;

FIG. 11C illustrates a transverse, cross sectional view of the balloon angioplasty catheter distal portion shaft of FIG. 11B, showing two lumens for carrying the inflation fluid and two for receiving the guide wires;

FIG. 11D illustrates a fragmentary, side view of the balloon angioplasty catheter of FIG. 11B disposed over two guide wires and carrying the stent of FIG. 2;

FIG. 12 illustrates a longitudinal cross sectional view of a bifurcated, stenosed, blood vessel region having a main branch and a first (main branch) and a second (side) branch continuing past the bifurcation, and having a first guide wire crossing the stenosis past the bifurcation and extending into the first branch;

FIG. 13 illustrates a longitudinal cross sectional view of a bifurcated, stenosed, blood vessel region of FIG. 12, having a guide catheter advanced to the bifurcation over the first guide wire;

FIG. 14 illustrates a longitudinal cross sectional view of the vessel region of FIG. 13, having the second guide wire advanced into the second (side) branch from the other lumen of the guide catheter;

FIG. 15 illustrates a longitudinal cross sectional view of the vessel region of FIG. 14, having the guide catheter being retracted over the placed guide wires;

FIG. 16 illustrates a longitudinal cross sectional view of the vessel region of FIG. 15, having a balloon angioplasty catheter carrying a Y-shaped stent being advanced out of a delivery sheath over the two guide wires to the bifurcation;

FIG. 17 illustrates a longitudinal cross sectional view of the vessel region of FIG. 16, having the main balloon for dilating the main channel and main branch advancing over the one guide wire and the side branch balloon for dilating the side branch being advanced over the other guide wire;

FIG. 18 illustrates a longitudinal cross sectional view of the vessel region of FIG. 17, having both balloons in place at the bifurcation; and

FIG. 19 illustrates a longitudinal cross sectional view of the vessel region of FIG. 18, having the balloons inflated at the bifurcation, expanding the stent into position against the vessel walls.

### DETAILED DESCRIPTION OF THE INVENTION

The present inventions provide apparatus and methods for providing medical treatment in a human body. The figures generally illustrate embodiments of apparatus including aspects of the present inventions. The particular exemplary embodiments of the apparatus according to the present inventions illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, variations of the apparatus and methods for treatment of stenosis at vessel bifurcations in the human body different from the illustrated embodiments may be encompassed by the appended claims.

The apparatus 20 and kits in accordance with the present inventions may include a guide catheter 22, a first guide wire 24, a second guide wire 26, and a balloon catheter 27. An expandable bifurcated stent 150 may be secured to a distal end of the balloon catheter 27. In one aspect, the balloon catheter 27 may include a mounted on at least a first balloon 94 and a second balloon 106 located generally on the distal portion 84 of the balloon catheter 27. The guide catheter 22 is used to position a first guide wire 24 in the main branch 170 of a vessel at a bifurcation 174 and to position a second guide wire 26 in the side branch 172 of a vessel at a bifurcation 174. After the first guide wire 24 and the second guide wire 26 are positioned, the guide catheter 22 is withdrawn from the patient.

The balloon catheter 27 will typically include a bifurcated stent 150 or set of stents 150 mounted on a bifurcated balloon or a balloon system. The balloon system may include a first balloon 94 and a second balloon 106 as generally illustrated in the figures. The balloon catheter 27 may have a first guide wire lumen 86 and a second guide wire lumen 88 extending along at least a portion of its length. The first guide wire lumen 86 is generally configured to slidably receive the first guide wire 24. The second guide wire lumen 88 is generally configured to slidably receive the second guide wire 26. After the guide catheter 22 is withdrawn from the patient, the bifurcated stent 150 is positioned within the bifurcation 174 by sliding the balloon catheter 27 with the bifurcated stent 150 mounted on the first balloon 94 and the second balloon 106 over the first guide wire 24 and the second guide wire 26. When the bifurcated stent 150 is positioned within the bifurcation 174, the first balloon 94 and the second balloon 106 are inflated to dilate the bifurcated stent 150, after which the first balloon 94 and the second balloon 106 deflated and the dual lumen balloon catheter 27 withdrawn from the patient.

As illustrated generally throughout the figures, the guide catheter 22 is used to position the first guide wire 24 into the main branch 170 of a vessel and to position the second guide wire 26 in the side branch 172 of a vessel at a bifurcation 174. The guide catheter 22 has a proximal portion 30 and a distal portion 32. The guide catheter 22 includes an elongate tubular body 28 having flexibility and stiffness characteristics that allow the elongate tubular body 28 to be maneuvered through various bodily lumen and positioned to deliver the first guide wire 24 into the main branch 170 of a vessel and positioned to deliver the second guide wire 26 into the side branch 172 of a vessel at a bifurcation 174. The elongate tubular body 28 defines at least a first guide wire lumen 34 and a second guide wire lumen 36 disposed in parallel within the guide tube 28. A centerline 40 may be defined by the first guide wire lumen 34 and a centerline 40 may be defined by the second guide wire lumen 36. The guide catheter 22 is generally configures so that the first centerline 40 of the first guide wire lumen 34 is generally parallel to the second centerline 41 of the second guide wire lumen 36. The first guide wire lumen 34 may have a constant radius about the first centerline 40, and the second guide wire lumen 36 may have a constant radius about the second centerline 41. The first guide wire lumen 34 is typically sized with a first lumen diameter which is greater than the first outside guide wire diameter of the first guide wire 24 to permit the first guide wire 24 to be slidably received within the first guide wire lumen 34. Similarly, the second guide wire lumen 36 is typically sized with a second lumen diameter which is greater than the second outside guide wire diameter of the second guide wire 26 to permit the second guide wire 26 to be slidably received within the second guide wire lumen 36.

The guide tube 28 may be configured in a variety of ways. For example, the guide tube 28 may be configured as a first tube 42 containing the first guide wire lumen 34 and a second tube 44 containing the second guide wire lumen 36 with the first tube 42 and the second tube 44 together to form the guide tube 28. The coupling may be, for example, in the form of a bond between the first tube 42 and the second tube 44. As another example, the first tube 42 and the second tube 44 may be collected together within a sheath 46 to form the guide tube 28. In yet another example, the guide tube 28 may be configured as a single tubular structure that includes the first guide wire lumen 34 and the second guide wire lumen 36. The guide tube 28 typically has an outside diameter small enough to be slidably received through the bodily lumen into which it is configured to position guide wires. Additional tubes or lumen may be included in any configuration.

The proximal portion 30 of the guide catheter 22 may include features to fascilitate the introduction of the first guide wire 24 into the first guide wire lumen 34 and the second guide wire 26 into the second guide wire lumen 36. These features may include a first guide wire introducer port 48 connected to the first guide wire lumen 34 of the guide tube 28 and a second guide wire introducer port 50 connected to the second guide wire lumen 36 of the guide tube 28. A manifold 52 having a first arm 54 and a second arm 56 may be positioned between the first guide wire introducer port 48 and the second guide wire introducer port 50 and the guide tube 28.

The distal portion 32 of the guide catheter 22 includes features configured to allow the first guide wire 24 and the second guide wire 26 to be introduced into the main branch 170 of a vessel and the side branch 172 of a vessel respectively at a bifurcation 174. A first exit port 62 is located at a distal end 58 of the first guide wire lumen 34 to allow the first guide wire 24 to pass out of the first guide wire lumen 34 to be introduced into the main branch 170 of a vessel at a bifurcation 174. A second exit port 64 is located at a distal end 60 of the second guide wire lumen 36 to allow the second guide wire 26 to pass out of the second guide wire lumen 36 to be introduced into the side branch 172 of a vessel at a bifurcation 174. The first exit port 62 is centered on the centerline 40 of the first guide wire lumen 34, and the second exit port 64 is centered on the centerline 40 of the second guide wire lumen 36.

When the guide tube 28 is generally configured as a first tube 42 containing the first guide wire lumen 34 and a second tube 44 containing the second guide wire lumen 36 with the first tube 42 and the second tube 44 coupled together to form the guide tube 28, the coupling may terminate proximal to the distal end 43 of the first tube 42 and proximal to the distal end 45 of the second tube 44. The terminus of the coupling between the first tube 42 and the second tube 44 may define a splay point 66. The first tube 42 is of substantially constant diameter from the splay point 60 to the distal end 43 of the first tube 42. The second tube 44 is of substantially constant diameter from the splay point 60 to the distal end 45 of the second tube 44. The distal portion 57 of the first tube 42 with respect to the splay point 66 may be splayed apart from the distal portion 59 of the second tube 44 with respect to the splay point 66. The length of the distal portion 57 of the first tube 42 with respect to the splay point 66 may either be substantially the same as or may vary from the length of the distal portion 59 of the second tube 44 with respect to the splay point 66. The distal portion 57 of the first tube 42 with respect to the splay point 66 may be placed into the main branch 170 of a vessel, and the distal portion 59 of the second tube 44 with respect to the splay point 66 may be placed into the side branch 172 of a vessel. This, in turn, may facilitate the placement of the first guide wire 24 into the main branch 170 of a vessel and may facilitate the placement of the second guide wire 26 into the side branch 172 of a vessel at a bifurcation 174.

Radiopaque marker bands 68 may be attached to the guide tube 28 generally proximate the distal portion 32 of the guide tube 28. The first tube 42 and the second tube 44 may be differentially marked with radiopaque marker bands 68 positioned on the first tube 42 and the second tube 44 distally from the splay point 66. Differential marking may be accomplished by, for example, marking the first tube 42 with one radiopaque marker band 68 and marking the second tube 44 with two radiopaque marker bands 68.

The first tube 42 and the second tube 44 may be configured so that the distal end 45 of the second tube 44 is positioned proximal to the distal end 43 of the first tube 42, so that the second exit port 64 is positioned proximal to the first exit port 62. Any bond or sheathing continues to the distal end 45 of the second tube 44, or the first tube 42 and the second tube 44 are otherwise configured to act in a unitary fashion from the proximal portion 30 of the guide tube 28 to the distal end 45 of the second tube 45. Configuring the guide catheter 22 so that the second exit port 64 is proximal to the first exit port 62 may facilitate placement of the first guide wire 24 into the main branch 170 of a vessel and may facilitate placement of the second guide wire 26 into the side branch 172 of a vessel at a bifurcation 174.

Radiopaque marker bands 68 may be placed proximate the distal end 43 of the first tube 24 and the distal end 45 of the second tube 44. Distal end 45 may be differentially marked from distal end 43 by, for example, placing one radiopaque marker band 68 proximate distal end 43 and placing two radiopaque marker bands 68 proximate distal end 45.

A first guide wire 24 may thus extend from first guide wire introducer port 48, through the first guide wire lumen 34, and through the first exit port 62, such that a distal portion of the first guide wire 24 may be placed into the main branch 170 of a vessel at a bifurcation 174. A second guide wire 26 may extend in a similar manner from second guide wire introducer port 50, through the second guide wire lumen 36, and through the second exit port 64, such that a distal portion of the second guide wire 26 may be placed into the side branch 172 of a vessel at a bifurcation 174.

The first guide wire 24 is sized so as to be slidably receivable within the first guide wire lumen 34, and the second guide wire 26 is sized to be slidably receivable within the second guide wire lumen 36. The first guide wire 24 and the first guide wire lumen 34 may differ in diameter from the second guide wire 26 and the second guide wire lumen 36.

The first guide wire 24 and the second guide wire 26 must have sufficient structural strength to guide the dual lumen balloon catheter 27. The first guide wire 24 and the second guide wire 26 may be made of stainless steel or other materials well known in the art.

The apparatus 20 of the present invention may also include a dual lumen balloon catheter 27, which has a delivery tube 80 configured as an elongate tubular body with a proximal portion 82 and a distal portion 84, and inflation source 90 within. The delivery tube 80 may be configured in sections having different mechanical characteristics that may facilitate passage of the delivery tube 80 through various bodily lumen.

A first distal tube 92 can extend from the distal portion 84 of the delivery tube 80. The first distal tube 92 may have an inflatable first balloon 94 secured thereto, with the first balloon 94 having an interior 96 and a first balloon length 98. The first distal tube 92 may have a first guide wire lumen 86 therethrough for receiving the first guide wire 24, and a first inflation lumen 122 in fluid communication with both the interior 96 of the first balloon 94 and with the inflation source 90 within the delivery tube 80.

A second distal tube 104 can extend from the distal portion 84 of the delivery tube 80, the second distal tube 104 having an inflatable second balloon 106 secured thereto, the second balloon 106 having an interior 96 and a second balloon length 108. The second distal tube 104 may have a second guide wire lumen 88 therethrough for receiving the second guide wire 26, and a second inflation lumen 128 in fluid communication with both the interior 96 of the second balloon 106 and with the inflation source 90 in the delivery tube 80.

The first balloon 94 has a first proximal balloon region 100 and a first distal balloon region 102. The second balloon 106 has a second proximal balloon region 110 and a second distal balloon region 112. The first balloon 94 and the second balloon 106 may be disposed, at least in part, in a substantially side by side relation with respect to each other. The first balloon length 98 and the second balloon length 108 may be substantially equal, or, the first balloon length 98 may be greater than the second balloon length 108. The first proximal balloon region 100 may be positioned proximal to the second proximal balloon region 110.

Some embodiments may include an inflatable third balloon 114 having an interior 96 and a third balloon length 116. The third balloon 114 has a third distal balloon region 120 and a third proximal balloon region 118. The third balloon 114 may be disposed on the first distal tube 92 such that the third balloon distal region 120 is proximal to the first proximal balloon region 100. The first guide wire lumen 86 and the second guide wire lumen 88 pass through the third balloon 114, and an inflation lumen, such as the first inflation lumen 122, is in fluid communication with both the interior 96 of the third balloon 114 and the inflation source 90. The first balloon 94, the second balloon 106, and the third balloon 114 may be configured as inflatable balloons, inflatable envelopes, or other dilation devices well known to those skilled in the art. Inflation may include filling the balloon with fluid or otherwise activating the balloon to expand in ways also recognized by those skilled in the art.

The inflation source 90 may include a first main inflation lumen 124 in fluid communication with both the first inflation lumen 122 and with the second inflation lumen 128. Alternatively, the inflation source 90 may include a first main inflation lumen 124 and a second main inflation lumen 130. The first main inflation lumen 124 is in fluid communication with the first inflation lumen 122, the first inflation lumen extending at least to the first proximal balloon region 100 so that the interior 96 of the first balloon 94 is in fluid communication with the first main inflation lumen 124. The second main inflation lumen 130 is in fluid communication with the second inflation lumen 128, the second inflation lumen 128 extending at least to the second proximal balloon region 110 so that the interior 96 of the second balloon 106 is in fluid communication with the second main inflation lumen 130.

The apparatus 20 of the present invention may also include an expandable bifurcated stent 150. The bifurcated stent 150 is a unitary device positionable in the bifurcation 174 of a blood vessel such that, when expanded, the bifurcated stent 150 supports the blood vessel walls throughout the region proximate the bifurcation 174. The bifurcated stent 150 includes at least an expandable main tube 152, an expandable first tube 154, and an expandable branch tube 156. The proximal portion 158 of the first tube 154, the proximal portion 160 of the branch tube 156, and the distal portion 162 of the main tube 152 are joined together through a joinder portion 164 such that the bifurcated stent 150 assumes a substantially Y-shape when deployed at a bifurcation 174 of a vessel.

The bifurcated stent 150 may be mounted to the first balloon 94, to the second balloon 106, and, in some embodiments, to the third balloon 114. For example, the main tube 152 may be configured to be carried over the third balloon 114, the first tube 154 may be configured to be carried over the first balloon 94, and the branch tube 156 may be configured to be carried over the second balloon 106, when the first balloon 94, the second balloon 106, and the third balloon 114 are in an uninflated state. Further details of a bifurcated stent which may be incorporated into kits and apparatus in accordance with the present inventions are disclosed in U.S. patent application Ser. No. 11/049,323 entitled BIFURCATED STENTING APPARATUS AND METHODS the disclosure of which is hereby incorporated by reference in its entirety.

One method includes advancing a guide catheter 22 to the vicinity of the bifurcation 152, where the guide catheter 22 has at least a first guide wire lumen 34 and a second guide wire lumen 36 disposed in a side by side relationship to each other. A first guide wire 24 may be distally advanced out of the first guide wire lumen into the main branch 170 of a vessel. A second guide wire 26 may be distally advanced out of the second guide wire lumen 36 into the side branch 172 of the vessel. In some methods, the guide catheter 22 may be advanced with the guide catheter 22 carrying either or both the first guide wire 24 and the second guide wire 26 through some regions, and with the guide catheter 22 being advanced over a more distally positioned first guide wire 24 and/or second guide wire 26 through other regions.

The method may include inserting at least one of the distal portion 57 of the first tube 42 or the distal portion 59 of the second tube 44 into main branch 170 or the side branch 172 of a vessel at a bifurcation 174. One of the distal portion 57 or distal portion 59 may extend further distally than the other of distal portion 57 or distal portion 59, with the method including advancing the distally further distal portion further distally past the bifurcation 174.

The first tube 42 and the second tube 44 may be configured such that the distal end 45 of the second tube 44 is positioned proximal to the distal end 43 of the first tube 42, so that the second exit port 64 is positioned proximal to the first exit port 62. Then, the method may include placing the dual distal end 43 of the first tube 42 and the distal end 45 of the second tube 44 with respect to the bifurcation 174 to facilitate placing the first guide wire 24 into the main branch 170 of a vessel and placing the second guide wire 26 into the side branch 172 of a vessel at a bifurcation 174.

Following placing the first guide wire 24 into the main branch 170 and the second guide wire 26 into the side branch, the guide catheter 22 may be withdrawn from the body over the first guide wire 24 and the second guide wire 26.

The method may include mounting an expandable bifurcated stent 150 to at least a first balloon 94 and a second balloon 106 portion of a dual lumen balloon catheter 27. The method continues by advancing the dual lumen balloon catheter 27 to the bifurcation 174 over the first guide wire 24 and the second guide wire 26, thereby advancing at least a first balloon 94 over the first guide wire 24 to at least the main branch 170 and thereby advancing a second balloon 106 over the second guide wire 26 to at least the side branch 172 at a bifurcation 174. The method may then involve placing at least the first balloon 94 in the main branch 170 and placing at least the second balloon 106 in at least the side branch 172 at the bifurcation. Inflating or otherwise activating the at least the first balloon 94 and the second balloon 106 may dilate the bifurcated stent 150 mounted to at least the first balloon 94 and the second balloon 106 and may dilate the respective vessel regions.

Some methods may include a third balloon 114 in which case the method encompasses advancing the third balloon 114 over at least the first guide wire 24 to at least the main branch 170 and then dilating a portion of the bifurcated stent 150 and a portion of the main branch 170 by inflating or otherwise activating the third balloon 114.

Then, the method proceeds by deflating the first balloon 94, the second balloon 106, and, if included, the third balloon 114, and withdrawing the dual lumen balloon catheter 27.

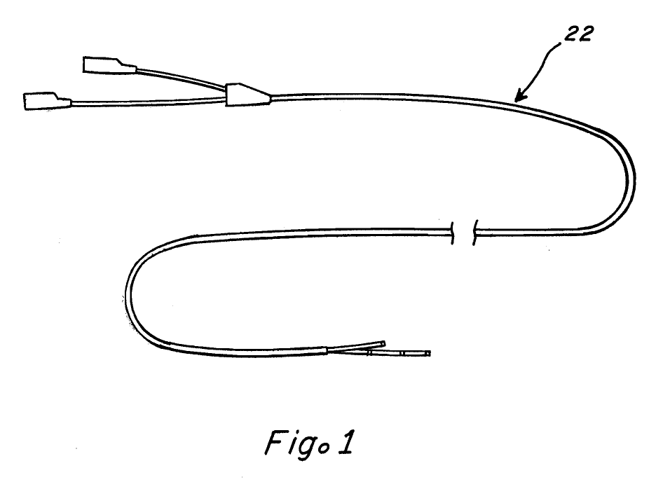

FIG. 1 illustrates an exemplary embodiment of a guide catheter 22 in accordance with the present inventions. FIG. 2 illustrates an exemplary embodiment of a kit in accordance with the present inventions. The kit may include a guide catheter 22, a first guide wire 24, and a second guide wire 26. In certain aspect, the kit may also include a balloon catheter 27.

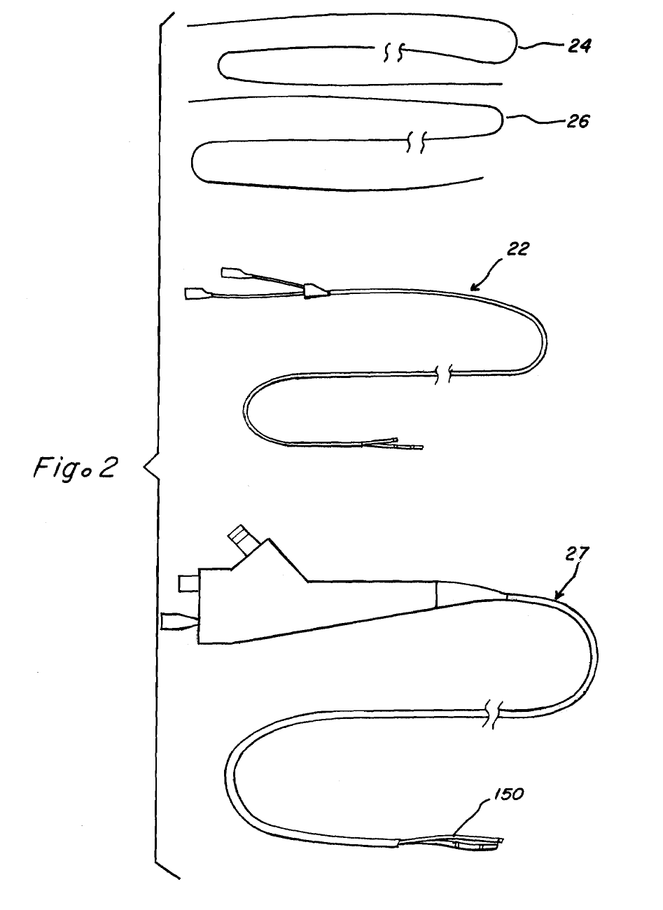

FIG. 3 illustrates an exemplary embodiment of a proximal portion 30 of a guide catheter 22. This Figure illustrates the proximal portion 35 of the guide tube 28 coupled to a manifold 52 having a first arm 54 and a second arm 56. A first introducer tube 47 having a first guide wire introducer port 48 and a second introducer tube 49 having a second guide wire introducer port 50 are shown coupled to the first arm 54 of the manifold 52 and the second arm 56 of the manifold 52, respectively. The first guide wire introducer port 48, the first introducer tube 47, the manifold 52, and the guide tube 28 are configured so that the first guide wire 24 may be introduced into the first guide wire introducer port 48, through the first introducer tube 47, through the manifold 52, and into the first guide wire lumen 34 within the guide tube 28. The second guide wire introducer port 50, the second introducer tube 49, the manifold 52, and the guide tube 28 are configured so that the second guide wire 26 may be introduced into the second guide wire introducer port 50, through second introducer tube 49, and into the second guide wire lumen 36 within the guide tube 28.

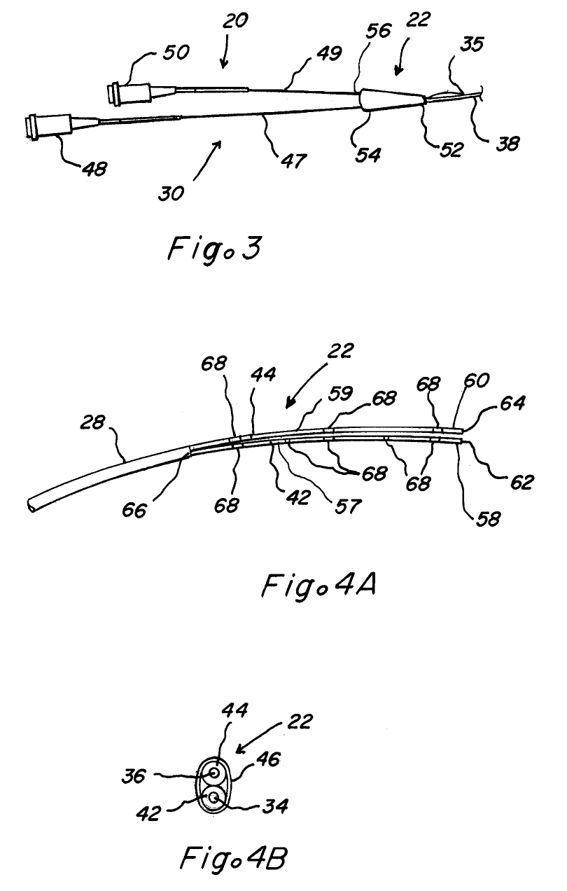

FIGS. 4A and 4B illustrate an exemplary configuration for a distal portion 32 of guide catheter 22. As illustrated, guide tube 28 is generally configured as a first tube 42 defining the first guide wire lumen 34 and a second tube 44 defining the second guide wire lumen 36 with the first tube 42 and the second tube 44 coupled together to form the guide tube 28 by a sheath 46. In the embodiment of FIG. 4A, the coupling between the first tube 42 and the second tube 44 terminates proximal to the distal end 43 of the first tube 42 and proximal to the distal end 45 of the second tube 44 thereby defining a splay point 66. The distal portion 57 of the first tube 42 with respect to the splay point 66 may be splayed apart from the distal portion 59 of the second tube 44 with respect to the splay point 66. The distal portion 57 of the first tube 42 with respect to the splay point 66 may be placed into the main branch 170 of a vessel. A first exit port 62 is located at a distal end 58 of the first guide wire lumen 34 within the first tube 42 to allow the first guide wire 24 to pass out of the first guide wire lumen 34 to be introduced into the main branch 170 of a vessel at a bifurcation 174. The distal portion 59 of the second tube 44 with respect to the splay point 66 may be placed into the side branch 172 of a vessel. A second exit port 64 is located at a distal end 60 of the second guide wire lumen 36 within the second tube 44 to allow the second guide wire 26 to pass out of the second guide wire lumen 36 to be introduced into the side branch 172 of a vessel at a bifurcation 174. The first tube 42 and the second tube 44 may extend the same distance from the splay point 66. In one aspect, it may follow that the first exit port 62 and the second exit port 64 may be positioned at substantially the same distance from the splay point 66 as generally illustrated in FIG. 4A. Radiopaque marker bands 68 may be attached to the guide tube 28 generally proximate the distal portion 32 of the guide tube 28. As illustrated in FIG. 4A, the first tube 42 and the second tube 44 may be differentially marked with radiopaque marker bands 68 positioned on the first tube 42 and the second tube 44 distally from the splay point 66.

FIGS. 5A and 5B illustrate another exemplary configuration for a distal portion 32 of guide catheter 22. As illustrated, guide tube 28 is configured as a first tube 42 defining the first guide wire lumen 34 and a second tube 44 containing the second guide wire lumen 36 with the first tube 42 and the second tube 44 coupled together to form the guide tube 28 by a sheath 46. In the embodiment of FIG. 5A, the coupling between the first tube 42 and the second tube 44 terminates proximal to the distal end 43 of the first tube 42 and proximal to the distal end 45 of the second tube 44 thereby defining a splay point 66. The distal portion 57 of the first tube 42 with respect to the splay point 66 may be splayed apart from the distal portion 59 of the second tube 44 with respect to the splay point 66. The distal portion 57 of the first tube 42 with respect to the splay point 66 may be placed into the main branch 170 of a vessel. A first exit port 62 is located at a distal end 58 of the first guide wire lumen 34 within the first tube 42 to allow the first guide wire 24 to pass out of the first guide wire lumen 34 to be introduced into the main branch 170 of a vessel at a bifurcation 174. The distal portion 59 of the second tube 44 with respect to the splay point 66 may be placed into the side branch 172 of a vessel. A second exit port 64 is located at a distal end 60 of the second guide wire lumen 36 within the second tube 44 to allow the second guide wire 26 to pass out of the second guide wire lumen 36 to be introduced into the side branch 172 of a vessel at a bifurcation 174. The first tube 42 and the second tube 44 may extend different distances from the splay point 66. In one aspect, it may follow that the first exit port 62 and the second exit port 64 may be positioned at divergent distances from the splay point 66. As is generally illustrated in FIG. 5A, first tube 42 may extend a shorter distance from splay point 66 than second tube 44 which can permit the second tube 42 to extend through the main bodily lumen past the branching bodily lumen before the second tube 44 is guided into the branching bodily lumen. Radiopaque marker bands 68 may be attached to the guide tube 28 generally proximate the distal portion 32 of the guide tube 28. As illustrated in FIG. 5A, the first tube 42 and the second tube 44 may be differentially marked with radiopaque marker bands 68 positioned on the first tube 42 and the second tube 44 distally from the splay point 66.

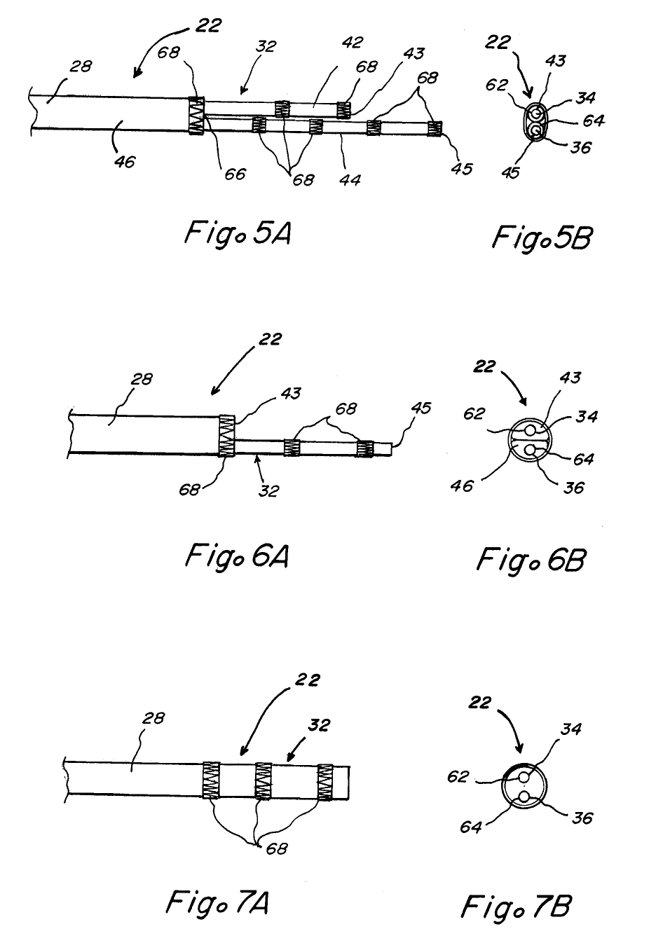

FIGS. 6A and 6B illustrate another exemplary configuration for a distal portion 32 of guide catheter 22. Guide catheter 22 is generally configured as an elongated tubular body 28 defining a first guide wire lumen 34 and a second guide wire lumen 36. In the embodiment of FIG. 6A, the distal end of the elongated tubular body 28 is configured such that a first exit port 62 of a first guide lumen 34 is located at a position proximal to a second exit port 64 of a second guide wire lumen 36. The first guide lumen 34 and the second guide lumen may extend different distances along the elongated body 28. In one aspect, it may follow that the first exit port 62 and the second exit port 64 may be positioned at divergent locations along the elongated body 28. As is generally illustrated in FIG. 6A, the first exit port 62 may be positioned proximal to the second exit port 64 which can permit the second exit port 64 to be extended through the main bodily lumen past the branching bodily lumen before the first exit port is guided into a position adjacent to or proximal to the branching bodily lumen. Radiopaque marker bands 68 may be attached to the guide tube 28 generally proximate the distal portion 32 of the guide tube 28.

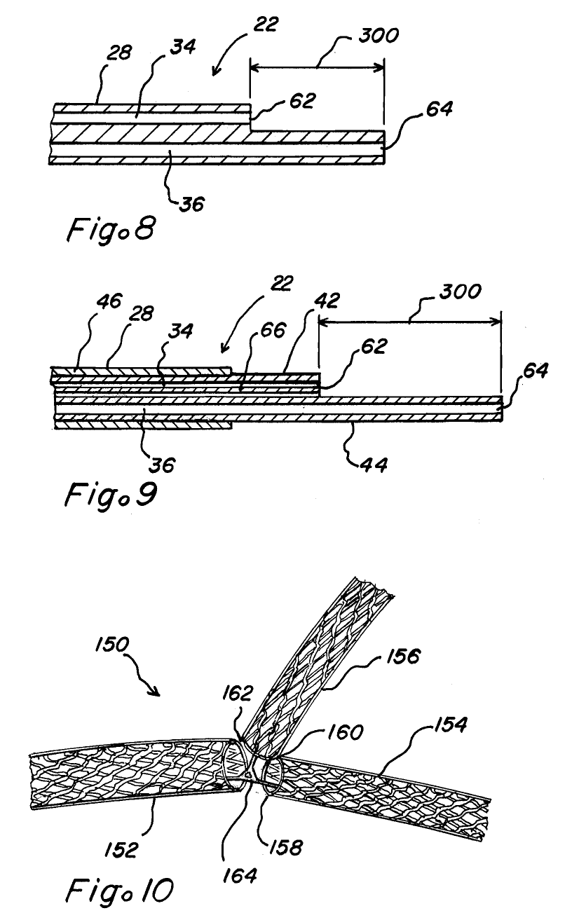

FIGS. 7A and 7B illustrate another exemplary configuration for a distal portion 32 of guide catheter 22. Guide catheter 22 is generally configured as an elongated tubular body 28 defining a first guide wire lumen 34 and a second guide wire lumen 36. In the embodiment of FIG. 7A, the distal end of the elongated tubular body 28 is configured such that a first exit port 62 of a first guide lumen 34 is positioned adjacent to a second exit port 64 of a second guide wire lumen 36 at a distal end of elongated tubular body 28. The first guide lumen 34 and the second guide lumen may extend the same distance along the elongated body 28. In one aspect, it may follow that the first exit port 62 and the second exit port 64 may be positioned at the same location along the elongated body 28. As is generally illustrated in FIG. 7A, the first exit port 62 and the second exit port 64 are positioned at the distal end of elongated tubular body 28 which can permit the first guide wire 24 and second guide wire 26 to exit the guide catheter 22 at its distal end. Radiopaque marker bands 68 may be attached to the guide tube 28 generally proximate the distal portion 32 of the guide tube 28.

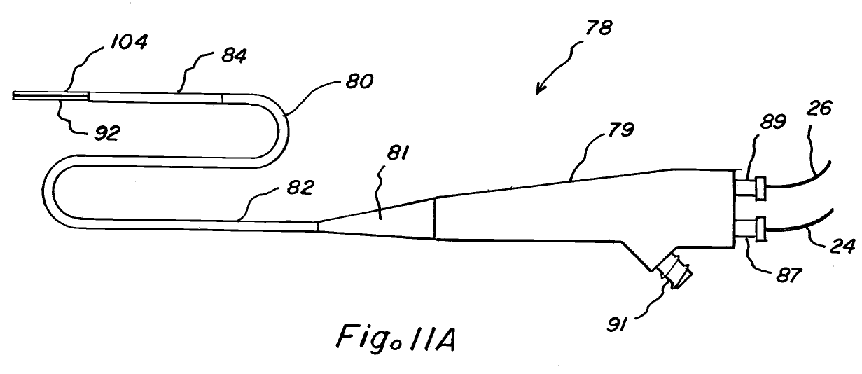

FIG. 8 illustrates a partial transverse cross-section at a distal end of a guide catheter 22 similar to the embodiments illustrated in FIGS. 6A and 6B. Guide catheter 22 is generally configured as a unitary elongated tubular body 28 defining a first guide wire lumen 34 and a second guide wire lumen 36. The distal end of the elongated tubular body 28 is configured such that a first exit port 62 of a first guide lumen 34 is located at a position proximal to a second exit port 64 of a second guide wire lumen 36. Particularly, the second exit port 64 is positioned distally a distance 300 from the first exit port 62.

FIG. 9 illustrates a partial transverse cross-section at a distal end of a guide catheter 22 similar to the embodiments illustrated in FIGS. 5A and 5B. As illustrated, guide tube 28 is configured as composite structure having a first tube 42 defining the first guide wire lumen 34 and a second tube 44 containing the second guide wire lumen 36 with the first tube 42 and the second tube 44 coupled together to form the guide tube 28 by a sheath 46. The coupling between the first tube 42 and the second tube 44 terminates proximal to the distal end 43 of the first tube 42 and proximal to the distal end 45 of the second tube 44 thereby defining a splay point 66. The distal portion 57 of the first tube 42 with respect to the splay point 66 may be splayed apart from the distal portion 59 of the second tube 44 with respect to the splay point 66. The first tube 42 and the second tube 44 may extend different distances from the splay point 66. In one aspect, it may follow that the first exit port 62 and the second exit port 64 may be positioned at divergent distances from the splay point 66. As is generally illustrated in FIG. 9, first tube 42 may extend a shorter distance from splay point 66 than second tube 44 which can permit the second tube 42 to extend through the main bodily lumen past the branching bodily lumen before the second tube 44 is guided into the branching bodily lumen. Particularly, the second exit port 64 is positioned distally a distance 300 from the first exit port 62.

FIG. 10 illustrates an embodiment of a bifurcated stent 150 that may be placed in a vessel at a bifurcation 174. The bifurcated stent 150 may include a main tube 152 that may be coupled to each of a first tube 154 and a branch tube 156. The distal portion 162 of the main tube 152, the proximal portion 158 of the first tube 154, and the proximal portion 160 of the branch tube 156 may be coupled at joinder portion 164, as illustrated in the Figure, to form a substantially Y-shaped bifurcated stent 150. The bifurcated stent 150 is generally configured as a mesh of stainless steel, Nitinol, and other materials well known to those skilled in the art.

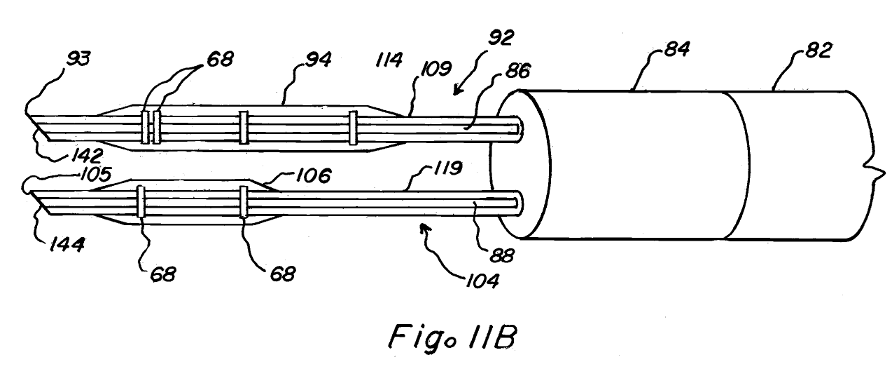

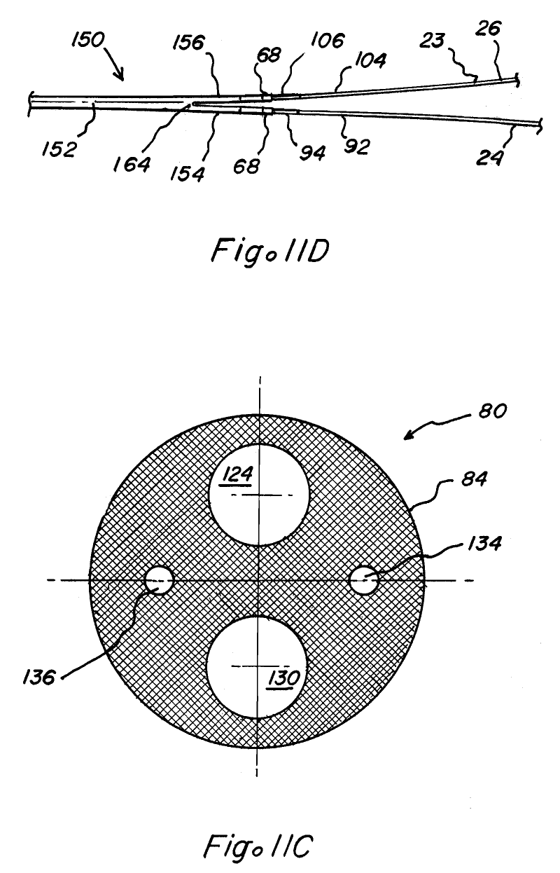

FIG. 11A illustrates a dual lumen balloon catheter 27, which may be used for placing the bifurcated stent 150 at a bifurcation 174 and for dilating the bifurcated stent 150 after placement. The embodiment illustrated in FIG. 11A includes a hub 79 extending through a strain relief 81 to a delivery tube 80 having a proximal portion 82 and further to a distal portion 84. The hub 79 may be formed of polycarbonate, and the strain relief 81 may be formed of Poly Ether Block Amide (e.g. PEBAX), which may have a Shore D hardness of about 35D in some embodiments. The proximal portion 82 may be formed of a braided material, for example braided polyimide, well known to those skilled in the art. The distal portion 84 may be formed of a softer material, for example, PEBAX having a Shore D hardness of about 72D in order to be more flexible than the proximal portion 82. Both the proximal portion 82 and the distal portion 84 may have an outer diameter of less than about 0.080 inch, or about 0.065 inch, in some embodiments.

The distal portion 84 extends further to two distinct distal tubes, a first distal tube 92 and a second distal tube 104, as illustrated in FIG. 11A. The first distal tube 92 and the second distal tube may carry the at least the first balloon 94 and the second balloon 106 and the bifurcated stent 150.

In the embodiment of FIG. 11A, the hub 79 includes a first guide wire port 87 configured with respect to the hub 79 to allow the first guide wire 24 to be removed from the balloon catheter 27, and a second guide wire port 89 configured with respect to the hub 79 to allow the second guide wire 26 to be removed from the balloon catheter 79. An inflation port 91 may also be included in the hub 79. The inflation port 91 may be connected to a balloon inflation device, as would be recognized by those skilled in the art. The balloon inflation device can be an Indeflater device, having a pressure indicator.

FIG. 11B illustrates the distal portion 84 of the delivery tube 80 in greater detail, in a highly diagrammatic fashion, including first distal tube 92 and second distal tube 104 having a second shaft 119. The distal tubes can be formed of high density polyethylene and have a first guide wire lumen 86 and a second guide wire lumen 88. In some embodiments, the distal shafts 109 and 119 can have an outer diameter of less than about 0.030 inch, or about 0.024 inch among other diameters which will vary with the particular application. The distal shaft guide wire lumens 110 and 120 may have an inside diameter of less than about 0.025 inch, or about 0.018 inch, in some embodiments among other diameters which will vary with the particular application. A first balloon 94 may be seen as well as a second inflatable envelope or balloon 106. In some embodiments, the first balloon 94 and the second balloon 106 may be made of Nylon (e.g. SDS) and may have a 16 atm rating. Radiopaque marker bands 68 may be seen, which may be formed of a Pt/Ir alloy (90%/10%) in some devices. The radiopaque marker bands 68 may vary to distinguish the first distal tube 92 from the second distal tube 104 as, for example, in the Figure, where the first distal tube 92 has double radiopaque marker bands 68. The first distal tube 90 extends to a first beveled tip 93 having a first distal guide wire port 142, and second distal tube 104 extends to a second beveled tip 105 having a second distal guide wire port 144, in some embodiments. The first beveled tip 93 may facilitate receiving the first guide wire 24 into the first guide wire lumen 86 through the first distal guide wire port 142. The second beveled tip 105 may facilitate receiving the second guide wire 26 respectively into the second distal tube guide wire lumen 88 through the second distal guide wire port 144

In some embodiments, the first distal tube 90 and second distal tube 104 may contain a first inflation lumen 122 (not shown) and a second inflation lumen 128 (not shown), respectively, that are separate from the first guide wire lumen 86 and from the second guide wire lumen 88. In other embodiments, the first guide wire lumen 86 may also serve as the first inflation lumen 122, and the second guide wire lumen 88 may also serve as the second inflation lumen 128.

A transverse cross-section of an embodiment of a distal portion 84 delivery tube 80 is illustrated in FIG. 11C. In the embodiment shown in FIG. 11C, the delivery tube 80 has a first main inflation lumen 124, a second main inflation lumen 130, a first main guide wire lumen 134, and a second main guide wire lumen 136.

FIG. 311D illustrates a bifurcated stent 150 disposed over the first balloon 94 and the second balloon 106 mounted on first distal tube 92 and second distal tube 104. The first guide wire 24 passes through the first distal tube 92, and the second guide wire 26 passes through the second distal tube 104, in the illustration. Bifurcated stent 150 includes main tube 152, first tube 154, and branch tube 156, joined at joinder portion 164. Both first guide wire 24 and second guide wire 26 may be seen passing through main tube 152. Beveled tips 93 and 105 are also shown.

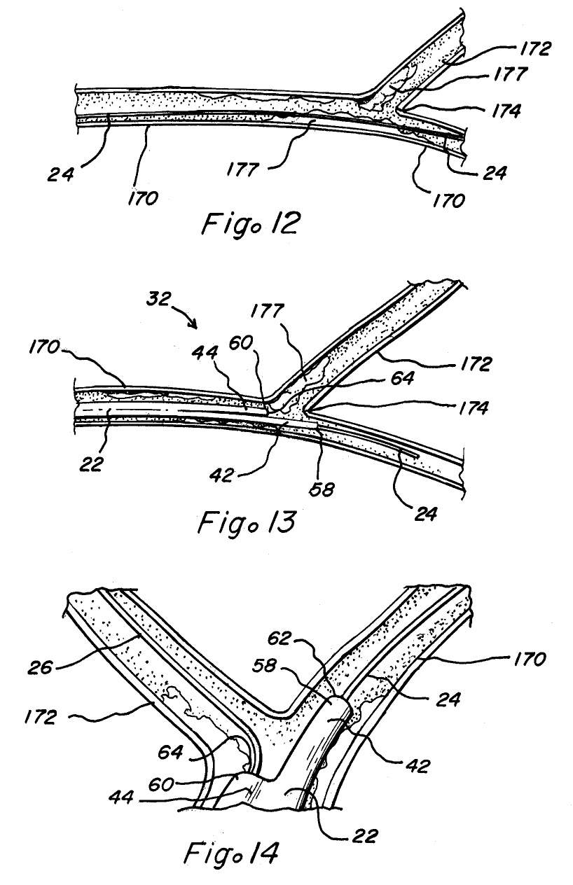

FIG. 12 illustrates an example of a stenosed, bifurcated vessel region including a main portion 170 extending through the bifurcation 174 and a side branch 172. Plaques 177 may be seen partially occluding the vessels. First guide wire 24 may be seen having been advanced through the main branch 170 past bifurcation 174.

FIG. 13 illustrates the distal portion 32 of the guide catheter 22 being advanced over first guide wire 100. In this illustration, the first tube 42 is positioned in the main branch 170 such that the distal end 58 and first exit port 62 are distal of the bifurcation 174. In the embodiment illustrated in FIG. 5, the second tube 44 has been clipped back such that distal end 60 and second exit port 64 are proximal of distal end 58 and first exit port 62. This allows the distal end 60 and second exit port 64 to be positioned just proximal to the side branch 172 in the bifurcation 174, as illustrated.

FIG. 14 illustrates an embodiment of the guide catheter 22. In this embodiment, the first tube 42 is positioned in the main branch 170 such that the distal end 58 and first exit port 62 are distal of the bifurcation 174 and the distal end 60 and second exit port 64 are positioned just proximal to the side branch 172. First guide wire 24 extends from the first exit port 62 into the main branch 170 distally from the bifurcation 174. The second guide wire 26 has been advanced out of the second exit port 64 into the side branch 172.

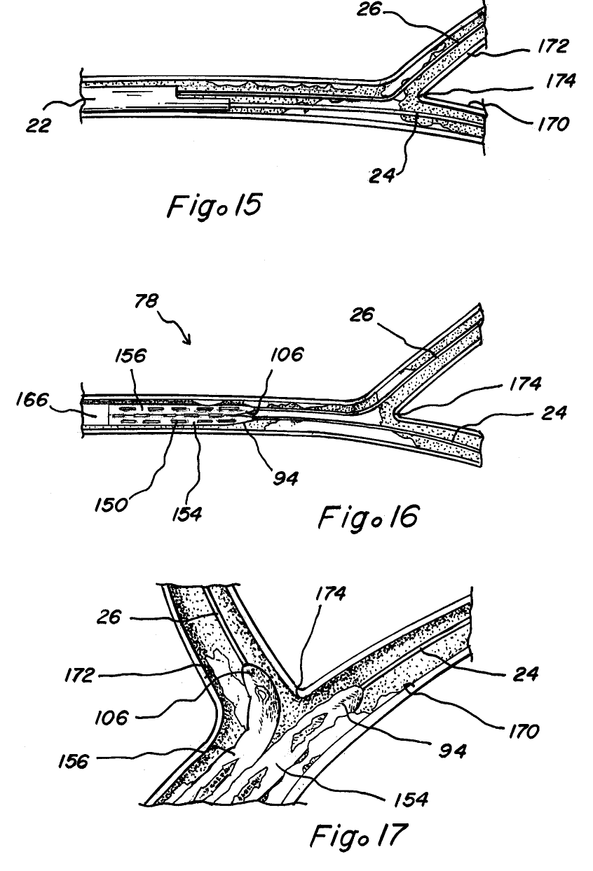

After the first guide wire 24 has been place in the main branch 170 and the second guide wire 26 has been placed in the side branch 172, the guide catheter 22 may be retracted over the placed first guide wire 24 and second guide wire 26, as illustrated in FIG. 15. In this Figure, the guide catheter 22 is shown as having been moved proximal to the bifurcation 174.

Delivery of the bifurcated stent 150 to the bifurcation 174 is illustrated in FIG. 16. In the embodiment illustrated in this Figure, the bifurcated stent 150 is mounted to the first balloon 94 and to the second balloon 106. The first balloon 94 carries the main tube 152 and the first tube 154 and is advanced over the first guide wire 24. The branch tube 156 is mounted to the second balloon 106, which is advanced over the second guide wire 26. In some embodiments, the balloons are delivered from a delivery sheath 166. The first balloon 94 and the second balloon 106 may be joined proximally or at an intermediate location and configured to separate when deployed at the bifurcation 174. The main tube 152 (not visible) is still located within sheath 160, carried on a more proximal portion of first balloon 94.

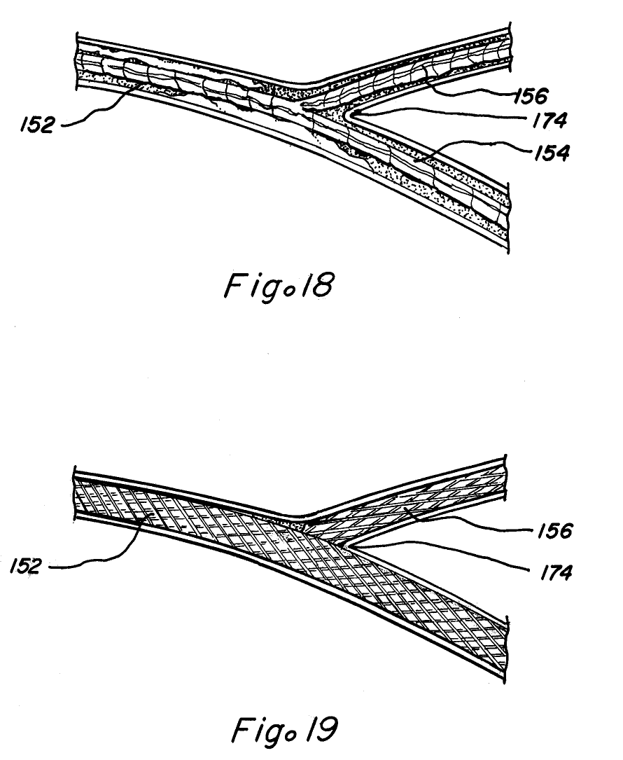

FIG. 17 illustrates first balloon 94 advancing over first guide wire 24 to carry first tube 154 into main branch 170, and second balloon 106 advancing over second guide wire 26 to carry branch tube 156 into side branch 172. The first balloon 94 has advanced to be located in the main branch 154 distal of the bifurcation 174 in the illustration.

FIG. 18 illustrates the bifurcated stent 150 including the main tube 152, the first tube 154, and the branch tube 156 positioned in the bifurcation 174 prior to inflation of the balloons to which the bifurcated stent 150 is mounted.

The main tube 152, the first tube 154, and the branch tube 156 expanded against the vessel walls by the inflation of the balloons within, in the illustration of FIG. 19. After successful dilation of the stenosed bifurcation 174, the balloons may be deflated and withdrawn, along with the guide wires. First balloon 94, in the illustrated embodiment, carries both main tube 152 and first tube 154, with first balloon length 98 being about twice second balloon length 108. In some embodiments of the invention, the first balloon length 98 can be at least 50 percent longer than the second balloon length 108. In other embodiments, their may be a waist region to separate the first balloon into proximal and distal regions, or into separate balloons, thus effectively forming a third balloon.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

I claim:

1. A balloon angioplasty catheter, comprising:
an elongate balloon having a proximal portion, a distal portion, and inflation source within;
a first distal tube extending from the balloon distal portion, the first distal tube having a first inflatable envelope secured thereto, the first inflatable envelope having an interior and a first envelope length, the first tube further having a guide wire lumen and a first inflation lumen in fluid communication with both the first envelope interior and with the inflation source in the elongate balloon; and
a second distal tube extending from the balloon distal portion, the second distal tube having a second inflatable envelope secured thereto, the second inflatable envelope having an interior and a second envelope length, the second tube further having a guide wire lumen and a second inflation lumen in fluid communication with both the second envelope interior and with the inflation source in the elongate balloon;
where the first and second inflatable envelopes are disposed at least in part in a substantially side by side relation to each other, and where the first envelope is substantially longer than the second envelope.

2. The catheter of claim 1, in which the first envelope has a distal portion and a proximal portion, further comprising an expandable tubular stent having a first tubular portion disposed over the first envelope distal portion, a second tubular portion disposed over the second envelope, and a third tubular portion disposed over the first envelope proximal portion.

3. The catheter of claim 1, in which the stent third tubular portion is coupled to the stent first and second tubular portions.

4. The catheter of claim 1, in which the second balloon envelope length is less than two thirds the first balloon envelope length.

5. The catheter of claim 1, in which the second balloon envelope length is less than half the first balloon envelope length.

6. The catheter of claim 1, in which the balloon inflation source includes a first main inflation lumen in fluid communication with both the first and second distal tube inflation lumens and extending to the balloon proximal region.

7. The catheter of claim 1, in which the balloon inflation source includes a first main inflation lumen in fluid communication with the first distal tube inflation lumen and extending to the balloon proximal region, and a second main inflation lumen in fluid communication with the second distal tube inflation lumen and extending to the balloon proximal region

8. The catheter of claim 1, in which the balloon includes a first main guide wire lumen in fluid communication with the first distal tube guide wire lumen and extending to the balloon proximal region, and a second main guide wire lumen in fluid communication with the second distal tube guide wire lumen and extending to the balloon proximal region.

9. A method for treating a bifurcated vessel region in a human body, the bifurcated vessel region having a main region splitting at a bifurcation into at least a first and a second region disposed distal of the main region, where the first region may be substantially aligned with the main region, the method comprising:
advancing a guide catheter to the vessel main region, the guide catheter having at least a first lumen and a second lumen disposed in a side by side relationship to each other, the guide catheter having a proximal portion and a distal portion;
advancing a first guide wire distally out of the first lumen and into the vessel first region;
advancing a second guide wire distally out of the second lumen and into the vessel second region.

10. The method of claim 1, in which the guide catheter distal portion includes a first distal tube having the first lumen within and a second distal tube having the second lumen within, the first and second distal tube being capable of being splayed apart, the method including inserting at least one of the first or second distal tubes into the respective first or second vessel regions.

11. The method of claim 2, in which one of the guide catheter first or second tubes extends further distally than the other of the first or second tubes, the method including advancing the distally further extending tube further distally past the bifurcation.

12. The method of claim 1, further including withdrawing the guide catheter from the body over the first and second guide wires, further including advancing a first dilation device over the first guide wire to at least the main region and a second dilation device over the second guide wire to at least the main region, and activating the first and second dilation devices to dilate the respective vessel regions.

13. The method of claim 4, in which the first and second dilation devices are joined at a joinder region to form a common dilation device having a first dilation portion disposed at least in part distal of the joinder and a second dilation portion disposed at least in part distal of the joinder region, the method further comprising dilating the first and second vessel regions using the first and second dilation devices.

14. The method of claim 5, in which the common dilation device includes a third dilation portion disposed proximal of the joinder region, the method further comprising dilating the main vessel region using the third dilation device.

15. The method of claim 6, in which the common dilation device is advanced over both the first and second guide wires.

16. The method of claim 6, in which the common dilation device includes a first guide wire lumen for receiving the first guide wire and a second guide wire lumen for receiving the second guide wire.

17. The method of claim 6, in which the common dilation device includes a common guide wire lumen for receiving the first guide wire and second guide wire.

18. The method of claim 6, in which at least two of the dilation devices carry a tubular stent, the method further comprising expanding the stents during the dilating and leaving the stents in place.

19. The method of claim 6, in which each of the dilation devices carry a stent tube, the method further comprising expanding the stent tubes during the dilating and leaving the stents in place in at least the first and second vessel regions.

20. A kit for treating a stenosed, bifurcated vessel region in a human body, the bifurcated region having a main vessel region branching distally past a bifurcation into a first branch region and a second branch region, the kit comprising:
a first guide wire and a second guide wire;
a guide catheter including a first elongate balloon having a first guide wire lumen within sized to slidably receive the first guide wire and having a second guide wire lumen within sized to slidably receive the second guide wire lumen;
a balloon catheter device including an second elongate balloon having a proximal portion and a distal portion, the distal portion including a first inflatable balloon having a first guide wire lumen therethrough for receiving the first guide wire, and a second inflatable balloon having a second guide wire lumen therethrough for receiving the second guide wire.

21. The kit of claim 1, in which the first inflatable balloon includes a proximal balloon region and a distal balloon region, where the first balloon proximal balloon region extends further proximally then the second balloon.

22. The kit of claim 1, further comprising a third inflatable balloon disposed proximally of the first inflatable balloon, and having a third guide wire lumen therethrough for receiving the first and second guide wires.

23. The kit of claim 3, in which the third guide wire lumen is a single lumen for receiving the first and second guide wires within.

24. The kit of claim 3, further comprising a stent device including a first expandable tubular portion sized to be carried over the first balloon when the first balloon is in an uninflated state, a second expandable tubular portion sized to be carried over the second balloon when the second balloon is in an uninflated state, and a third expandable tubular portion sized to be carried over the third balloon, when the third balloon is in an uninflated state, where the first and second stent tubular portions are each joined proximally to the third stent tubular portion at a distal portion of the stent third tubular portion, such that the stent device has a substantially Y shape when deployed at the vessel bifurcation.

25. The kit of claim 2, further comprising a stent device including a first expandable tubular portion sized to be carried over the first balloon distal portion when the first balloon is in an uninflated state, a second expandable tubular portion sized to be carried over the second balloon when the second balloon is in an uninflated state, and a third expandable tubular portion sized to be carried over the first balloon proximal portion, when the first balloon is in an uninflated state, where the first and second stent tubular portions are each joined proximally to the third stent tubular portion at a distal portion of the stent third tubular portion, such that the stent device has a substantially Y shape when deployed at the vessel bifurcation.
